(19) United States
(12) Patent Application Publication
(10) Pub. No.: US 2025/0099528 A1
(43) Pub. Date: Mar. 27, 2025

(54) ORAL COMPOSITION

(71) Applicant: Toyo Shinyaku Co., Ltd., Fukuoka-shi (JP)

(72) Inventors: Hiroya TAKAHASHI, Saga (JP); Yoshifumi Koseki, Saga (JP)

(73) Assignee: Toyo Shinyaku Co., Ltd., Fukuoka-shi (JP)

(21) Appl. No.: 18/473,672

(22) Filed: Sep. 25, 2023

(51) Int. Cl. A61K 36/8998; A23L 33/105; A61K 31/198; A61K 38/01

(52) U.S. Cl. CPC A61K 36/8998; A23L 33/105; A61K 31/198; A61K 38/014

(57) ABSTRACT

An oral composition which contains a dark-green vegetable and one or more selected from an amino acid, a peptide, and a protein. A method for producing an oral composition, the method containing mixing a dark-green vegetable and one or more selected from an amino acid, a peptide, and a protein.

### BACKGROUND OF THE INVENTION

### Technical Field

The present invention relates to an oral composition containing a dark-green vegetable and one or more selected from an amino acid, a peptide, and a protein.

### Description of Related Art

Protein is one of the main components of the human body. Hormones and enzymes as well as muscles, internal organs, and skin are composed of proteins, and proteins are essential for the biological activity. Proteins are not absorbed in the intestines as they are but are absorbed after being degraded into amino acids. The absorbed amino acids are transported to the liver, where the amino acids are re-synthesized into proteins and play various roles in the body.

In the human body, not only synthesis of proteins but also degradation thereof constantly occur. When the amount of degradation exceeds the amount of synthesis, the protein amount in the body decreases, and there are risks such as decline in muscle strength. Accordingly, appropriately taking proteins and amino acids is necessary for young people to old people to maintain and promote the health. Moreover, as stated above, proteins are absorbed in the body after being degraded into amino acids, and thus efficient absorption of amino acids is important in addition to appropriate intake of proteins and amino acids.

With these points as background, development of a material which promotes amino acid absorption is desired. Although a method for increasing amino acid absorption with a medicament (Patent Literature 1) and the like have been developed so far, the method and the like are not readily available to healthy individuals who wish to supplement proteins and amino acids in their daily lives.

### CITATION LIST

### Patent Literature

[Patent Literature 1] U.S. Pat. No. 6,734,170B

### SUMMARY OF INVENTION

### Technical Problem

The present invention has been made in view of the above problems and is intended to provide an oral composition containing a dark-green vegetable and one or more selected from an amino acid, a peptide, and a protein.

### Solution to Problem

The present inventors have found out, in the process of developing a material which promotes amino acid absorption, that the efficiency of amino acid absorption is increased by taking one or more selected from an amino acid, a peptide, and a protein in combination with a dark-green vegetable and thus have completed the present invention.

Specifically, the present invention is as follows.
(1) An oral composition containing a dark-green vegetable and one or more selected from an amino acid, a peptide, and a protein.
(2) The oral composition according to (1), wherein the dark-green vegetable is a dry powder.
(3) The oral composition according to (2), wherein the dry powder of the dark-green vegetable is a crushed powder or a juice powder of the dark-green vegetable.
(4) The oral composition according to any one of (1) to (3), wherein the dark-green vegetable is a green leaf of at least one plant selected from oats, kale, ocarina, mulberry, Angelica keiskei, wormwood, Peucedanum japonicum, Kuma Bamboo Grass, spinach, Swiss chard, and celery.
(5) The oral composition according to any one of (1) to (4), wherein the dark-green vegetable is young barley grass.
(6) The oral composition according to any one of (1) to (5), containing leucine.
(7) The oral composition according to any one of (1) to (6), containing one or more selected from a collagen and a collagen peptide.
(8) The oral composition according to any one of (1) to (7), wherein a content of the dark-green vegetable is 0.1% by weight or more based on the oral composition.
(9) The oral composition according to any one of (1) to (8), containing dietary fiber, wherein a content of the dietary fiber is 0.05% by weight or more based on the oral composition.
(10) The oral composition according to any one of (1) to (9), wherein a total amount of the amino acid, the peptide, and the protein is 0.05% by weight or more based on the oral composition.
(11) The oral composition according to any one of (1) to (10), wherein a total amount of the amino acid, the peptide, and the protein is 1 part by weight or more based on 100 parts by weight of the dark-green vegetable.
(12) The oral composition according to any one of (1) to (11), wherein a content of leucine is 0.1 parts by weight or more based on 100 parts by weight of the dark-green vegetable.
(13) The oral composition according to any one of (1) to (12), containing dietary fiber, wherein a total amount of the amino acid, the peptide, and the protein is 1 part by weight or more based on 100 parts by weight of the dietary fiber.
(14) The oral composition according to any one of (1) to (13), containing dietary fiber, wherein a content of leucine is 0.1 parts by weight or more based on 100 parts by weight of the dietary fiber.
(15) The oral composition according to any one of (1) to (14), which is a powder food.
(16) A method for producing an oral composition, including mixing a dark-green vegetable and one or more selected from an amino acid, a peptide, and a protein.
(17) A method for improving amino acid absorption by administering a dark-green vegetable and one or more selected from an amino acid, a peptide, and a protein.

### Advantageous Effects of Invention

According to the present invention, because a dark-green vegetable and one or more selected from an amino acid, a peptide, and a protein are contained, an oral composition exhibiting excellent amino acid absorption can be provided.

### DESCRIPTION OF EMBODIMENTS

The present disclosure is explained in detail in the following.

### [1. Dark-Green Vegetables]

The dark-green vegetables of the present disclosure relate to vegetables having green color, specifically include green leaves of plants such as oats, kale, ocarina, mulberry, Angelica keiskei, wormwood, Peucedanum japonicum, Kuma Bamboo Grass, spinach, Swiss chard, celery, cabbage, colored green, lettuce, carrot, and tea, and vegetables such as pepper, goya, Japanese mustard spinach, green onion, Chinese chive, Broccoli, and peas. Parts of dark-green vegetables used in the present disclosure are not limited as long as it includes parts having green color, and for example, leaves, stem, and berries can be used.

As dark-green vegetables of the present disclosure, plants belonging to any family can be used, and for examples plants of Granmineae, Brassicaceae, Amaranthaceae, Apiaceae, Convolvulaceae, Moraceae, Asteraceae, and Chenopodiaceae can be used. From the viewpoint of having excellent amino acid absorption, and the viewpoint of comprising nutrient components in abundance, plants of Granmineae, Brassicaceae, Amaranthaceae, Apiaceae are more preferable, plants of Granmineae, Brassicaceae, Amaranthaceae are particularly preferable, and plants of Granmineae are most preferable.

As dark-green vegetables of the present disclosure, plants belonging to any genus can be used, and for examples plants of Granmineae Hordeum, Granmineae Sasa, Brassicaceae Brassica, Amaranthaceae Spinacia, Apiaceae Angelica, Apiaceae Peucedanum, Apiaceae Apium, Convolvulaceae Ipomoea, Moraceae Morus, Asteraceae Artemisia, Chenopodiaceae Beta, etc. can be used. From the viewpoint of having particularly excellent amino acid absorption, and the viewpoint of comprising nutrient components in abundance, plants of Granmineae Hordeum, Granmineae Sasa, Brassicaceae Brassica, Amaranthaceae Spinacia are more preferable, plants of Granmineae Hordeum, Granmineae Sasa, Brassicaceae Brassica, Amaranthaceae Spinacia are more preferable, and plants of Granmineae Hordeum are most preferable.

### [2. Green Leaves of Specific Plant]

As dark-green vegetables of the present disclosure, any dark-green vegetables can be used. However, from the viewpoint of having particularly excellent amino acid absorption, and the viewpoint of comprising nutrient components in abundance, it is particularly preferable to use green leaves of specific plants as described in the following as dark-green vegetables. Specific plants of the present disclosure mean at least one plant selected from oats, kale, ocarina, mulberry, Angelica keiskei, wormwood, Peucedanum japonicum, Kuma Bamboo Grass, spinach, Swiss chard and celery. Only one type can be used, but two or more types can be also used. From the viewpoint of having particularly excellent amino acid absorption, and the viewpoint of comprising nutrient components in abundance, among green leaves of specific plants, green leaves of oats are particularly preferable, and green leaves of barley (young barley grass) are most preferable.

Green leaves of specific plants of the present disclosure relate to plant body including leaf part of the specific plant, and can include leaves, as well as stem, or other parts. Since it takes effort to remove stem from the aerial part at the time of harvest, and the stem part also comprises nutrient components such as insoluble dietary fiber, etc. in abundance, from the viewpoint of reducing manufacturing cost and the viewpoint of alibility, it is preferable to comprise stem together with leaves.

### <Oats>

Oats of the present disclosure mean a collective term of gramineous plant having similar appearance, such as barley, wheat, rye, oat, etc. As oats of the present disclosure, barley is particularly preferable.

Barley (Hordeum vulgare L.) is said to be native of Central Asia, is a poaceous annual or biennial grass plant belonging to Granmineae Hordeum. As barley used in the present disclosure, breed is not particularly limited. Barley of any breed can be used, and even wild species or crossbred can be used. For example, two-row barley, six-row barley, naked barley etc. can be exemplified, and these can be used by combining one or more breeds.

Green leaves of oats can be any one harvested at any harvest season, and are not particularly limited. For example, when oats are barley, it is preferable to use those harvested before mature stage, specifically those harvested from initiation of tillering stage to before initiation of ear emergence (young barley grass). Specifically, it is preferable to use young barley grass harvested from barley having a height of 10 cm or more, preferably about 10 to 90 cm, particularly preferably about 20 to 80 cm, best of all about 30 to 70 cm.

### <Kale>

Kale of the present disclosure means a plant of Brassicaceae Brassica, which scientific name is Brassica oleracea var. acephala. Breed of kale is not particularly limited, and various types of kale such as Kitchen Kale, Tree Kale, Bush Kale, Marrow Kale, Collard, Green leaf kale, etc. can be used. Green leaves of kale can be any one harvested at any harvest season, and are not particularly limited.

### <Ocarina>

Ocarina of the present disclosure means a plant of Convolvulaceae Ipomoea, which scientific name is Ipomea batatas. Ocarina is not particularly limited as long as it is one generally called sweet potato. Breed of ocarina is not particularly limited, and for example, breeds such as Suio, Joy White, Kogane-sengan, Shiroyutaka, Satsuma starch, Ayamurasaki can be exemplified. Among these, Suio having a high polyphenol content is preferable. Green leaves of ocarina can be any one harvested at any harvest season, and are not particularly limited. Tip part of stem and leaf (young stem and leaf) is preferable, and young stem and leaf in a state maintaining yellow-tinged green color is further preferable.

### <Mulberry>

Mulberry of the present disclosure means a plant of Moraceae Morus (genus Morus). Species of mulberry are not particularly limited, and Morus alba, Morus australia, Morus lhou, Morus laevigata, Morus tiliaefolia, Morus boninensis, Morus serrata, etc. can be used. Green leaves of mulberry can be any one harvested at any harvest season, and are not particularly limited.

<Angelica keiskei>

Angelica keiskei of the present disclosure means a plant of Apiaceae Angelica. Breed of Angelica keiskei is not particularly limited and can be appropriately used. Green leaves of Angelica keiskei can be any one harvested at any harvest season, and are not particularly limited.

### <Wormwood>

Wormwood of the present disclosure means perennial plant of Asteraceae Artemisia (genus Artemisia). Species of wormwood are not particularly limited, and Artemisia indica, Artemisia absinthium, Artemisia dracunculus, Nitrof wormwood, Artemisia congesta, Artemisia capillaris, Artemisia japonica, Artemisia scoparia, Artemisia apiacea, Artemisia annua, Artemisia keshkeana, Artemisia pedenculosa, Artemisia trifucata, Artemisia arctica, Artemisia sinanensis, Artemisia glomerata, Artemisia lacinata, Artemisia stelleriana, Artemisia iwayomogi, Artemisia feddei, Artemisia gilvescens, Artemisia dubia, Artemisia monophylla, Artemisia unalaskensis, Artemisia koidzumii, Artemisia stolonifera, Artemisia momiyamae, Artemisia ruburipes, Artemisia montana, Artemisia indica, Artemisia schmidtiana, Artemisia kitadakensis, etc. can be used. Among these, Artemisia indica is particularly preferable. Green leaves of Artemisia indica can be any one harvested at any harvest season, and are not particularly limited.

<Peucedanum japonicum>

Peucedanum japonicum of the present disclosure means a plant of Apiaceae Pternopetalum. Peucedanum is called Danboufu, Chomeiso, Chomeigusa, Chomifusa, Bofu, Sakuna, Ububasafuna, Chomigusa, Botanbofu depending on the region. Breed of Peucedanum japonicum is not particularly limited, and can be appropriately used. Green leaves of Peucedanum japonicum can be any one harvested at any harvest season, and are not particularly limited.

### <Kuma Bamboo Grass>

Kuma Bamboo Grass of the present disclosure means a plant of Sasa beitchii (genus Sasa). Species of Kuma Bamboo Grass are not particularly limited, and Sasa veitchii, Sasa kurilensis, Sasamorpha borealis, Sasa senanensis, Sasa palmata, Sasa niponica, etc. can be used. Green leaves of Kuma Bamboo Grass can be any one harvested at any harvest season, and are not particularly limited.

### <Spinach>

Spinach of the present disclosure means a plant of Amaranthaceae Spinacia, which scientific name is Spinacia oleracea. Breed of spinach is not particularly limited, and can be appropriately used. Green leaves of spinach can be any one harvested at any harvest season, and are not particularly limited.

<Swiss chard>

Swiss chard of the present disclosure means a plant of Chenopodiaceae Beta, which scientific name is Beta vulgaris. In Japan, it is also called Fudanso. Breed of Swiss chard is not particularly limited, and can be appropriately used. Green leaves of Swiss chard can be any one harvested at any harvest season, and are not particularly limited.

### <Celery>

Celery of the present disclosure means a plant of Apiaceae Apium, which scientific name is Apium graveolens. Breed of celery is not particularly limited, and can be appropriately used. Green leaves of celery can be any one harvested at any harvest season, and are not particularly limited.

### [3. Dry Powder of Dark-Green Vegetables]

As dark-green vegetables used for the oral composition of the present disclosure, for example, dry powder of dark-green vegetables can be used. Dry powder of dark-green vegetable of the present disclosure means those made by processing dark-green vegetables and dried and powderized. Examples of dry powder of dark-green vegetables include those made by crushing dark-green vegetables and dried and powderized (crushed powder of dark-green vegetables), those made by drying and powderizing juice of dark-green vegetables (juice powder of dark-green vegetables), and those made by drying and powderizing extract of dark-green vegetables (extract powder of dark-green vegetables), but it is not limited to these. As dry powder of dark-green vegetables of the present disclosure, it is preferable to be crushed powder of dark-green vegetables from the viewpoint of having excellent amino acid absorption, the viewpoint of easiness of processing, storage, transport, etc. and the viewpoint of comprising dietary fiber in abundance.

As crushed powder of dark-green vegetables of the present disclosure, those obtained for example by combining drying treatment and crushing treatment can be used. Drying treatment and crushing treatment can be performed at the same time, or either one can be performed before the other one. However, it is preferable to first perform drying treatment, and then perform crushing treatment. According to need, one or more treatments selected from treatment such as blanching treatment and disinfection treatment can be performed in combination. The number of times for performing crushing treatment is not particularly limited, and it can be performed as one or more treatments, such as after performing coarse crushing treatment, performing fine crushing for crushing more finely.

Blanching treatment is a treatment to keep the green color of dark-green vegetables of the specific plant bright, and examples of a method of blanching treatment include hot-water treatment and steaming treatment.

Examples of hot-water treatment include a method of treating dark-green vegetables in hot water or moisture of for example 70 to 100° C., preferably 80 to 100° C., for 60 to 240 sec, preferably for 90 to 180 sec. As steaming treatment, an intermittent steaming treatment of repeating a treatment of steaming dark-green vegetables with moisture and a treatment of cooling at normal pressure or under pressure is preferable.

Disinfection treatment is not particularly limited as long as it is a common disinfection treatment to a person skilled in the art. For example, it can be a treatment of killing microorganisms physically or chemically by using temperature, pressure, electromagnetic wave, drugs, etc.

When performing blanching treatment additionally to drying treatment and crushing treatment, it is preferable that blanching treatment is performed before drying treatment. Further, when performing disinfection treatment additionally to drying treatment and crushing treatment, it is preferable that disinfection treatment is performed after drying treatment, or before or after crushing treatment.

Drying treatment is not particularly limited, but examples include treatment of drying so that the water content of dark-green vegetables becomes 10% by weight or less, preferably 8% by weight or less.

Crushing treatment is not particularly limited, but examples include treatment of crushing plant bodies by an arbitrary method that a person skilled in the art commonly uses. When performing crushing treatment, it is preferable to perform the treatment by combining coarse crushing and fine crushing, from the viewpoint of increasing crushing efficiency, such as to make the particle size uniform, shortening the crushing time, etc.

As juice powder of dark-green vegetables of the present disclosure, for example, those obtained by concentrating juice of dark-green vegetables at low temperature to concentrate the solid content, and lyophilizing or spray drying the concentrated liquid can be used.

### [4. Amino Acid, Peptide, and Protein]

The composition of the present disclosure is characterized by containing one or more selected from an amino acid, a peptide, and a protein. As the amino acid, the peptide or the protein of the present disclosure, a synthetic material may be used, or a processed plant product or a processed animal product containing an amino acid, a peptide or a protein may be used. The amino acid, the peptide or the protein used for the present disclosure is preferably an amino acid, a peptide or a protein derived from an origin other than dark-green vegetables, particularly preferably an amino acid, a peptide or a protein derived from an origin other than green leaves of the specific plants above, and most preferably an amino acid, a peptide or a protein derived from an origin other than young barley grass.

### <Amino Acid>

The amino acid used for the present disclosure is not particularly restricted but is preferably one or more essential amino acids selected from tryptophan, leucine, lysine, valine, threonine, phenylalanine, methionine, isoleucine, and histidine from the viewpoint of excellent absorption and from the viewpoint of supplementing nutrients. Of the essential amino acids, from the viewpoint of excellent absorption in the body, one or more branched-chain amino acids selected from valine, leucine, and isoleucine are particularly preferable, and leucine is most preferable. In this regard, the amino acids in the present disclosure mean free amino acids and do not include amino acids constituting a protein.

### <Peptide>

The peptide used for the present disclosure is not particularly restricted, and for example, a soy peptide, a sardine peptide, a collagen peptide or the like can be used. From the viewpoint of excellent absorption in the body, a collagen peptide is most preferable.

### <Protein>

The protein used for the present disclosure is not particularly restricted, and for example, an animal protein such as whey, casein, and collagen or a plant protein such as soybeans can be used. From the viewpoint of excellent absorption in the body, one or more proteins selected from soy protein, whey protein, casein, and collagen are particularly preferable, and collagen is most preferable.

### [5. Oral Composition]

### <Contents>

The oral composition of the present disclosure contains a dark-green vegetable. The dark-green vegetable content of the oral composition of the present disclosure is not particularly restricted but is, from the viewpoint of especially excellent amino acid absorption, preferably 0.1% by weight or more in terms of dried weight, more preferably 1% by weight or more, further preferably 5% by weight or more, particularly preferably 10% by weight or more, and most preferably 30% by weight or more. The upper limit of the dark-green vegetable content may be, for example, 95% by weight or less or may be 90% by weight or less.

From the viewpoint of containing dietary fiber in abundance, the dark-green vegetable of the present disclosure is preferably crushed powder. When the dark-green vegetable is crushed powder, the dietary fiber content of the oral composition of the present disclosure is not particularly restricted but is, from the viewpoint of especially excellent amino acid absorption, preferably 0.05% by weight or more in terms of dried weight, more preferably 0.5% by weight or more, further preferably 2.5% by weight or more, particularly preferably 5% by weight or more, and most preferably 15% by weight or more. The upper limit of the dietary fiber content may be, for example, 50% by weight or less or may be 30% by weight or less.

The oral composition of the present disclosure contains one or more selected from an amino acid, a peptide, and a protein. The total amount of the amino acid, the peptide, and the protein in the oral composition of the present disclosure is not particularly restricted but is, from the viewpoint of especially excellent amino acid absorption, preferably 0.05% by weight or more in terms of dried weight, more preferably 0.5% by weight or more, further preferably 2.5% by weight or more, particularly preferably 5% by weight or more, and most preferably 15% by weight or more. The upper limit of the total amount of the amino acid, the peptide, and the protein may be, for example, 50% by weight or less or may be 30% by weight or less.

The branched amino acid or leucine content of the composition of the present disclosure is not particularly restricted but is, from the viewpoint of especially excellent amino acid absorption, preferably 0.01% by weight or more in terms of dried weight, more preferably 0.05% by weight or more, further preferably 0.5% by weight or more, particularly preferably 2% by weight or more, and most preferably 5% by weight or more. The upper limit of the branched amino acid content may be, for example, 50% by weight or less or may be 10% by weight or less.

### <Weight Ratios>

In the oral composition of the present disclosure, the weight ratio of the dark-green vegetable and the amino acid, the peptide, and the protein is not particularly restricted, but from the viewpoint of especially excellent amino acid absorption, the amino acid, the peptide, and the protein are contained preferably in 1 part by weight or more in total in terms of dried weight based on 100 parts by weight of the dark-green vegetable, more preferably in 10 parts by weight or more, further preferably in 50 parts by weight or more, particularly preferably in 70 parts by weight or more, and most preferably in 80 parts by weight or more. The upper limit may be, for example, 1000 parts by weight or less or may be 500 parts by weight or less.

In the oral composition of the present disclosure, the weight ratio of the dark-green vegetable and the branched amino acid or leucine is not particularly restricted, but from the viewpoint of especially excellent amino acid absorption, the branched amino acid or leucine is contained preferably in 0.1 parts by weight or more in terms of dried weight based on 100 parts by weight of the dark-green vegetable, more preferably in 1 part by weight or more, further preferably in 10 parts by weight or more, particularly preferably in 15 parts by weight or more, and most preferably in 20 parts by weight or more. The upper limit may be, for example, 1000 parts by weight or less or may be 500 parts by weight or less.

In the oral composition of the present disclosure, the weight ratio of the dietary fiber and the amino acid, the peptide, and the protein is not particularly restricted, but from the viewpoint of especially excellent amino acid absorption, the amino acid, the peptide, and the protein is contained preferably in 1 part by weight or more in total in terms of dried weight based on 100 parts by weight of the dietary fiber, more preferably in 20 parts by weight or more, further preferably in 100 parts by weight or more, particularly preferably in 150 parts by weight or more, and most preferably in 200 parts by weight or more. The upper limit may be, for example, 1000 parts by weight or less or may be 500 parts by weight or less.

In the oral composition of the present disclosure, the weight ratio of the dietary fiber and the branched amino acid or leucine is not particularly restricted, but from the viewpoint of especially excellent amino acid absorption, the branched amino acid or leucine is contained preferably in 0.1 parts by weight or more in terms of dried weight based on 100 parts by weight of the dietary fiber, more preferably in 5 parts by weight or more, further preferably in 10 parts by weight or more, particularly preferably in 20 parts by weight or more, and most preferably in 30 parts by weight or more. The upper limit may be, for example, 1000 parts by weight or less or may be 500 parts by weight or less.

### <Form or the Like>

The form of the oral composition of the present disclosure is not particularly limited, and the oral composition can be in any form. Examples thereof include forms such as powder (including granules), tablets, liquid, paste, and capsules. When the present disclosure is in powder form (powder form such as powder and granules), excellent dispersibility is exhibited when mixed in a liquid such as water, and thus the present disclosure is particularly preferably in powder form.

The oral composition of the present disclosure may contain a component other than the dark-green vegetable, the amino acid, the peptide, and the protein, such as various excipients, lubricants, stabilizers, thickening agents, emulsifiers, colorants, and aromas.

The oral composition of the present disclosure can be used for foods, pharmaceutical products, or the like but is particularly preferably used as a food because the dark-green vegetable contains nutrient components in abundance.

### Examples

In the following, the present invention is explained in further detail with the Examples. However, the present invention is not limited to these Examples, and as long as the problems of the present invention can be solved, the present invention can have various embodiments.

### [1. Free Amino Acid Absorption-Promoting Action]

To assess the action of promoting free amino acid absorption of a dark-green vegetable, leucine permeability was measured by a test using Caco-2 cells.

### 1-1. Sample Preparation

The following materials were used as test substances.
(1) As a dark-green vegetable, crushed powder of young barley grass (containing 43% by weight of dietary fiber; manufactured by TOYO SHINYAKU Co., Ltd.) which was obtained by washing grass harvested before heading, then cutting, drying, and then crushing was used. The crushed powder of young barley grass in an amount of 24 mg was put into a tube, and after adding 1 mL of a buffer, the mixture was vortexed. Then, the supernatant was collected through centrifugation at 15,000×g for five minutes at room temperature. The supernatant was disinfected with a filter, then diluted and used as a test substance.
(2) As a branched-chain amino acid, L-leucine (manufactured by Nacalai Tesque, Inc.: 20327-46) was used. L-leucine in an amount of 13.1 mg was put into a tube and dissolved in 1 mL of a buffer added thereto (100 mM). The solution was disinfected with a filter, then diluted and used as a test substance. The final concentrations of the test substances are shown in Table 1.

TABLE 1

| | Comparative Example 1 | | Example 1 | |
|---|---|---|---|---|
| | (A) | (B) | (A) | (B) |
| Crushed Powder of | — | — | 2 | 2 |
| Young Barley Grass | | | | |
| L-Leucine | — | 0.131 | — | 0.131 |

Unit mg/mL

### 1-2. Cell Culture and Addition of Test Substances

(1) Human colon carcinoma-derived cells (Caco-2) were cultured using a 75-cm² flask in an incubator at 37° C. at 5% CO₂. The medium used was 10% FBS-DMEM.
(2) The medium in a volume of 600 μl was added to a 24-well plate, and the insert wells were transferred.
(3) The cells which were suspended by trypsin treatment in a volume of 100 μl were seeded from the 75-cm² flask to the insert wells to obtain a cell density of 4.0×10⁴ cells/well.
(4) The cells were pre-cultured in an incubator at 37° C. at 5% CO₂ for 14 days, and the medium was changed every two or three days during the pre-culture. When the medium was changed before the addition of the samples, the medium outside the insert wells (the wells at the basolateral side) was replaced with 1000 μl of 10% FBS-DMEM, and the medium inside the insert wells (the wells at the luminal side) was replaced with 350 μl of 10% FBS-DMEM.
(5) The electrical resistance values were measured with Millicell ERS-2, and the wells having a value of 1000Ω or more were used. The wells were grouped in such a manner that the averages of the electrical resistance values of the groups to which the test substances would be added became the same.
(6) After washing the outside of the insert wells three times and the inside three times each with 1000 μL of a buffer, the cells were transferred to a 24-well plate containing 1000 μL of the buffer. The buffer in a volume of 350 μL was added to the insert wells.
(7) The cells were cultured in a CO₂ incubator for 30 minutes and acclimated to the buffer.
(8) After removing 175 μL of the buffer inside the insert wells, 175 μL of test substance solutions prepared at the 2× concentration were added. The cells were cultured in a CO₂ incubator for two hours.
(9) After the culture, the entire amounts of the samples in the wells at the basolateral side were collected in 1.5-mL tubes.

### 1-3. Measurement of Leucine Concentrations by High Performance Liquid Chromatography

After dispensing 500 μL of the samples in the wells at the basolateral side collected in 1-2 (9) above in tubes, the samples were dried using a centrifugal evaporator. After further drying 400 μL of the samples in the same tubes, 200 μL of 100 mM HCl was added, and the samples were dissolved with ultrasonic waves for 10 minutes. The leucine concentrations of the obtained concentrated liquids were measured with high performance liquid chromatography. The measurement results were applied to the equation below, and thus the leucine permeability was calculated.

$$Leucine Permeability ( \% ) = \{ Leucine Concentration ( mM ) at Basolateral Side of ( B ) \times 1000 ( μL ) - Leucine Concentration ( mM ) at Basolateral Side of ( A ) \times 1000 ( μL ) \} / \{ Added Leucine Concentration at Luminal Side ( mM ) \times 350 ( μL ) \} \qquad [ Math . 1 ]$$

### 1-4. Test Results

The results are shown in Table 2. While the leucine permeability of Comparative Example 1, which contained leucine only, was 1.32%, that of Example 1, which contained leucine and the crushed powder of young barley grass, was 3.86%, which was about three times higher. From the results, it was found that amino acid absorption is promoted when crushed powder of young barley grass is administered together with leucine.

TABLE 2

| | Comparative | |
|---|---|---|
| | Example 1 | Example 1 |
| Leucine Permeability (%) | 1.32 | 3.86 |

### [2. Protein/Peptide Absorption-Promoting Action]

To examine whether amino acid absorption would be promoted by a dark-green vegetable also when a protein or a peptide was administered instead of the free amino acid, a test for assessing the absorption of collagen (a protein) was conducted. In this test, as an indicator of collagen absorption, the blood hydroxyproline concentrations were measured. Hydroxyproline is an amino acid constituting collagen. Because hydroxyproline accounts for about 11 to 14% of the amino acids constituting collagen and exists distinctively in collagen, hydroxyproline is generally used as an indicator for assessing collagen absorption.

### 2-1. Test Method

After giving MF feed (manufactured by Oriental Yeast Co., ltd.) to seven-week-old male Wistar rats (Kyudo Co., Ltd.) as a standard feed and acclimating the rats for one week, the rats were divided into three groups in such a manner that the average body weights of the groups became the same (n of each group was three to five). A test feed, a reference feed or the standard feed was given to the three groups. The compositions of the test feed and the reference feed are shown in Table 3. As the dark-green vegetable, crushed powder of young barley grass (containing 42% by weight of dietary fiber; manufactured by TOYO SHINYAKU Co., Ltd.) was used.

TABLE 3

| | Test Feed | Reference Feed |
|---|---|---|
| Milk Casein | 20% | 20% |
| Lard | 20% | 20% |
| Mineral Mixture | 4% | 4% |
| Vitamin Mixture | 1% | 1% |
| Cholesterol | 1% | 1% |
| Sodium Cholate | 0.25% | 0.25% |
| Crushed Powder of Young Barley Grass | 11.8% | — |
| β-Cornstarch | 41.95% | 53.75% |
| Total | 100% | 100% |

On the 28th day after starting free feeding, 5 g/kg of collagen (a mixture of pig-derived collagen: fish-derived collagen=1:1) was administered to the rats of the groups under fasting for 16 hours. Blood samples were taken from the abdominal vena cava of the rats just before starting the administration and two hours after the administration, and plasma samples were obtained. Subsequently, the samples were each separated with a cation exchange resin column, and the fluorescence intensities (348 nm and 450 nm) obtained by two-step reaction of oxidation by a sodium hypochlorite solution and emission of fluorescence by an ortho-phthalaldehyde solution were measured. The blood hydroxyproline concentrations of the samples were measured by HPLC (manufactured by JASCO Corporation).

### 2-2. Test Results

The results are shown in Table 4. The blood hydroxyproline amount of the test feed group was higher than that of the reference feed group. From the results, it was found that protein or peptide absorption is promoted with crushed powder of young barley grass.

TABLE 4

| Blood Hydroxyproline | Test Feed | Reference Feed | Standard Feed |
|---|---|---|---|
| Amount | Group | Group | Group |
| Before Starting | 74 | 74 | 74 |
| Administration | | | |
| Two Hours After | 620 | 476 | 369 |
| Administration | | | |

Unit μmol/l

### [3. Production of Oral Compositions Containing Dark-Green Vegetables, Amino Acids and the Like]

Raw materials were mixed as shown in Table 5 and then granulated by a fluidized-bed granulation using a granulator, and powder drinks in granule form of Examples 2-21 were produced. The obtained powder drinks all exhibited excellent amino acid absorption and excellent dispersibility when being suspended in water.

| | | Example | | | | | | | | | |
|---|---|---|---|---|---|---|---|---|---|---|---|
| | | 2 | 3 | 4 | 5 | 6 | 7 | 8 | 9 | 10 | 11 |
| Dark-Green | Crushed Powder of Young Barley Grass | 10 | 10 | 10 | 10 | 10 | 10 | 10 | 10 | 10 | |
| Vegetable | Juice Powder of Young Barley Grass | | | | | | | | | | 10 |
| | Crushed Powder of Kale | | | | | | | | | | |
| | Crushed Powder of Young Ocarina Leaves | | | | | | | | | | |
| | Crushed Powder of Mulberry Leaves | | | | | | | | | | |
| | Crushed Powder of Angelica keiskei | | | | | | | | | | |
| | Crushed Powder of Wormwood | | | | | | | | | | |
| | Crushed Powder of Peucedanum japonicum | | | | | | | | | | |
| | Crushed Powder of Kuma Bamboo Grass | | | | | | | | | | |
| | Crushed Powder of Spinach | | | | | | | | | | |
| | Crushed Powder of Swiss Chard | | | | | | | | | | |
| | Crushed Powder of Celery | | | | | | | | | | |
| Amino Acid | Leucine | 10 | | | | | | | | | 10 |
| | Valine | | 10 | | | | | | | | |
| | Isoleucine | | | 10 | | | | | | | |
| | Glycine | | | | 10 | | | | | | |
| Peptide | Collagen Peptide | | | | | 10 | | | | | |
| Protein | Collagen | | | | | | 10 | | | | |
| | Whey Protein | | | | | | | 10 | | | |
| | Casein | | | | | | | | 10 | | |
| | Soy Protein | | | | | | | | | 10 | |
| Other | Sucrose | 10 | 10 | 10 | 10 | 10 | 10 | 10 | 10 | 10 | 10 |
| | Reduced Maltose | 70 | 70 | 70 | 70 | 70 | 70 | 70 | 70 | 70 | 70 |
| Total | | 100 | 100 | 100 | 100 | 100 | 100 | 100 | 100 | 100 | 100 |
| | | Example | | | | | | | | | |
| | | 12 | 13 | 14 | 15 | 16 | 17 | 18 | 19 | 20 | 21 |
| Dark-Green | Crushed Powder of Young Barley Grass | | | | | | | | | | |
| Vegetable | Juice Powder of Young Barley Grass | | | | | | | | | | |
| | Crushed Powder of Kale | 10 | | | | | | | | | |
| | Crushed Powder of Young Ocarina Leaves | | 10 | | | | | | | | |
| | Crushed Powder of Mulberry Leaves | | | 10 | | | | | | | |
| | Crushed Powder of Angelica keiskei | | | | 10 | | | | | | |
| | Crushed Powder of Wormwood | | | | | 10 | | | | | |
| | Crushed Powder of Peucedanum japonicum | | | | | | 10 | | | | |
| | Crushed Powder of Kuma Bamboo Grass | | | | | | | 10 | | | |
| | Crushed Powder of Spinach | | | | | | | | 10 | | |
| | Crushed Powder of Swiss Chard | | | | | | | | | 10 | |
| | Crushed Powder of Celery | | | | | | | | | | 10 |
| Amino Acid | Leucine | 10 | 10 | 10 | 10 | 10 | 10 | 10 | 10 | 10 | 10 |
| | Valine | | | | | | | | | | |
| | Isoleucine | | | | | | | | | | |
| | Glycine | | | | | | | | | | |
| Peptide | Collagen Peptide | | | | | | | | | | |
| Protein | Collagen | | | | | | | | | | |
| | Whey Protein | | | | | | | | | | |
| | Casein | | | | | | | | | | |
| | Soy Protein | | | | | | | | | | |
| Other | Sucrose | 10 | 10 | 10 | 10 | 10 | 10 | 10 | 10 | 10 | 10 |
| | Reduced Maltose | 70 | 70 | 70 | 70 | 70 | 70 | 70 | 70 | 70 | 70 |

| | | | | | | | | | | |
|---|---|---|---|---|---|---|---|---|---|---|
| Total | 100 | 100 | 100 | 100 | 100 | 100 | 100 | 100 | 100 | 100 |

### INDUSTRIAL APPLICABILITY

The present invention relates to an oral composition which contains a dark-green vegetable and one or more selected from an amino acid, a peptide, and a protein and which exhibits excellent amino acid absorption and can be used as a food. Thus, its industrial applicability is high.

What is claimed is:

1. An oral composition comprising a dark-green vegetable and one or more selected from an amino acid, a peptide, and a protein.

2. The oral composition according to claim 1, wherein the dark-green vegetable is a dry powder.

3. The oral composition according to claim 2, wherein the dry powder of the dark-green vegetable is a crushed powder or a juice powder of the dark-green vegetable.

4. The oral composition according to claim 1, wherein the dark-green vegetable is a green leaf of at least one plant selected from oats, kale, ocarina, mulberry, Angelica keiskei, wormwood, Peucedanum japonicum, Kuma Bamboo Grass, spinach, Swiss chard, and celery.

5. The oral composition according to claim 1, wherein the dark-green vegetable is young barley grass.

6. The oral composition according to claim 1, comprising leucine.

7. The oral composition according to claim 1, comprising one or more selected from a collagen and a collagen peptide.

8. The oral composition according to claim 1, wherein a content of the dark-green vegetable is 0.1% by weight or more based on the oral composition.

9. The oral composition according to claim 1, comprising dietary fiber, wherein a content of the dietary fiber is 0.05% by weight or more based on the oral composition.

10. The oral composition according to claim 1, wherein a total amount of the amino acid, the peptide, and the protein is 0.05% by weight or more based on the oral composition.

11. The oral composition according to claim 1, wherein a total amount of the amino acid, the peptide, and the protein is 1 part by weight or more based on 100 parts by weight of the dark-green vegetable.

12. The oral composition according to claim 1, wherein a content of leucine is 0.1 parts by weight or more based on 100 parts by weight of the dark-green vegetable.

13. The oral composition according to claim 1, comprising dietary fiber, wherein a total amount of the amino acid, the peptide, and the protein is 1 part by weight or more based on 100 parts by weight of the dietary fiber.

14. The oral composition according to claim 1, comprising dietary fiber, wherein a content of leucine is 0.1 parts by weight or more based on 100 parts by weight of the dietary fiber.

15. The oral composition according to claim 1, which is a powder food.

16. A method for producing an oral composition, comprising mixing a dark-green vegetable and one or more selected from an amino acid, a peptide, and a protein.
